(19) United States
(12) Patent Application Publication
(10) Pub. No.: US 2002/0127653 A1
(43) Pub. Date: Sep. 12, 2002

(54) Hematopoietic signaling factor

(75) Inventors: Daniel R. Soppet, Centreville, VA (US); Steven M. Ruben, Olney, MD (US)

Correspondence Address: HUMAN GENOME SCIENCES INC, 9410 KEY WEST AVENUE, ROCKVILLE, MD, 20850

(73) Assignee: Human Genome Sciences, Inc., Rockville, MD

(21) Appl. No.: 09/996,606

(22) Filed: Nov. 30, 2001

Related U.S. Application Data
(63) Continuation of application No. 09/008,490, filed on Jan. 16, 1998, now abandoned.
(60) Provisional application No. 60/035,577, filed on Jan. 16, 1997.

(51) Int. Cl. C12P 21/02; C07H 21/04; C12N 5/06; C07K 14/52

(52) U.S. Cl. 435/69.5; 435/325; 435/320.1; 530/351; 536/23.5

(57) ABSTRACT

The present invention relates to a novel hematopoietic signaling factor (HSF) protein which is a member of the cytokine superfamily. In particular, isolated nucleic acid molecules are provided encoding the human HSF protein. HSF polypeptides are also provided as are vectors, host cells and recombinant methods for producing the same.

### CROSS-REFERENCE TO RELATED APPLICATIONS

This application hereby claims priority benefit to U.S. Appl. No. 60/035,577, filed Jan. 16, 1997, which disclosure is hereby incorporated by reference.

### BACKGROUND OF THE INVENTION

1. Field of the Invention

The present invention relates to a novel cellular signaling molecule. More specifically, isolated nucleic acid molecules are provided encoding a human hematopoietic signaling factor (HSF). HSF polypeptides are also provided, as are vectors, host cells and recombinant methods for producing the same. Also provided are diagnostic methods for detecting pathological disorders and therapeutic methods.

2. Related Art

Hematopoiesis is the production of mature blood cells involving a complex scheme of multilineage differentiation. Mature blood cells are derived from pluripotent hematopoietic stem cells which are typically present at very low frequencies (<1.0%) in hematopoietic tissues. The defining characteristics of hematopoietic stem cells are the capacity for extensive self-renewal and retention of multilineage differentiation potential, that is, the ability to reconstitute the hematopoietic system. Hematopoietic stem cells proliferate and differentiate to produce progenitor cells, which in turn form precursor cells, which differentiate to form mature blood cells.

During ontogeny, hematopoiesis moves from yolk sac to liver and spleen and then to the bone marrow (Travassoli, M., Blood Cells 17:269 (1991)). During early fetal life, hematopoiesis occurs within the liver and spleen. In the latter part of gestation, bone marrow spaces begin to develop and expand. Hematopoietic stem cells then migrate from the liver and spleen to the bone marrow occupying “niches” in the developing marrow (Zanjani et al., J. Clin. Invest. 89:1178 (1992)). Subsequently, hematopoiesis primarily occurs in the bone marrow (Gordon et al., Bone Marrow Transplant 4:335 (1989)).

There has been much interest in the ex vivo expansion of hematopoietic stem cells, particularly as an alternative to bone marrow transplantation (Edgington S. M., Biotechnology, 10:1099 (1992)). For instance, successful ex vivo expansion of primitive stem and progenitor cells would allow transplantations in situations where, using currently available technology, adequate amounts of bone marrow cannot be harvested from the patient.

It has been demonstrated that proliferation and differentiation of hematopoietic stem cells are regulated by a group of glycoproteins known as hematopoietic cytokines. Numerous investigations have focused on the ability of different combinations of these hematopoietic growth factors, or signal factors, to stimulate hematopoietic cell expansion (Meunch et al., Blood 81:3463 (1993); Bodine et al, Blood 79:913 (1992); Kobayashi et al., Blood 78:1947 (1991); Berstein et al., Blood 77:2316 (1991); Brandt et al., J. Clin. Invest. 86:932; and Moore et al., Proc. Natl. Acad. Sci. USA 84:7134 (1987)).

Because of the wide range of activities regulated by hematopoietic growth factors, their utility has been manifested in the fields of immunodeficiency diseases, autoimmune diseases, infectious diseases, hepatitis, nephritis, cancers, and bone marrow transplantations. In addition to hematopoietic growth factors, the antibodies of the factors and the receptors for the factors are also useful as diagnostic agents.

In view of the wide range of roles that hematopoietic cells play in physiologic and pathologic processes, there is a continuing need for the isolation and characterization of novel hematopoietic cell regulatory proteins.

### SUMMARY OF THE INVENTION

The present invention provides isolated nucleic acid molecules comprising a polynucleotide encoding the HSF polypeptide having the amino acid sequence shown in SEQ ID NO:2 or the amino acid sequence encoded by the cDNA clone deposited in a bacterial host as ATCC Deposit Number 97731 on Sep. 23, 1996.

The present invention also relates to recombinant vectors, which include the isolated nucleic acid molecules of the present invention, and to host cells containing the recombinant vectors, as well as to methods of making such vectors and host cells and for using them for production of HSF polypeptides or peptides by recombinant techniques.

The invention further provides an isolated HSF polypeptide having an amino acid sequence encoded by a polynucleotide described herein.

The invention further provides isolated antibodies that bind specifically to a HSF polypeptide having an amino acid sequence as described herein. Such antibodies are useful diagnostically or therapeutically as described below.

An additional aspect of the invention is related to a method for treating an individual in need of an increased level of HSF activity in the body comprising administering to such an individual a composition comprising a therapeutically effective amount of an isolated HSF polypeptide of the invention or an agonist thereof.

A still further aspect of the invention is related to a method for treating an individual in need of a decreased level of HSF activity in the body comprising, administering to such an individual a composition comprising a therapeutically effective amount of an HSF antagonist.

### BRIEF DESCRIPTION OF THE FIGURES

FIGS. 1A and 1B show the nucleotide (SEQ ID NO:1) and deduced amino acid (SEQ ID NO:2) sequences of HSF. The protein has a predicted leader sequence of about 26 amino acid residues (underlined) and a deduced molecular weight of about 38 kDa. It is further predicted that amino acid residues from about 27 to about 379 (about 1 to about 353 in SEQ ID NO:2) constitute the mature HSF protein.

FIG. 2 shows the regions of similarity between the amino acid sequences of the HSF protein and the Xenopus lfng protein (SEQ ID NO:3).

FIG. 3 shows an analysis of the HSF amino acid sequence. Alpha, beta, turn and coil regions; hydrophilicity and hydrophobicity; amphipathic regions; flexible regions; antigenic index and surface probability are shown. In the “Antigenic Index—Jameson-Wolf” graph, amino acid residues about 1 to about 10, about 27 to about 52, about 82 to about 116, about 120 to about 132, about 138 to about 163, about 172 to about 207, about 217 to about 248, about 283 to about 292, about 319 to about 330, and about 337 to about 377 in FIG. 1 correspond to the shown highly antigenic regions of the HSF protein. These highly antigenic fragments in FIG. 1 correspond to the following fragments, respectively, in SEQ ID NO:2: amino acid about −26 to about −17, about 1 to about 26, about 56 to about 90, about 94 to about 106, about 112 to about 137, about 146 to about 181, about 191 to about 222, about 257 to about 266, about 293 to about 304, and about 311 to about 351.

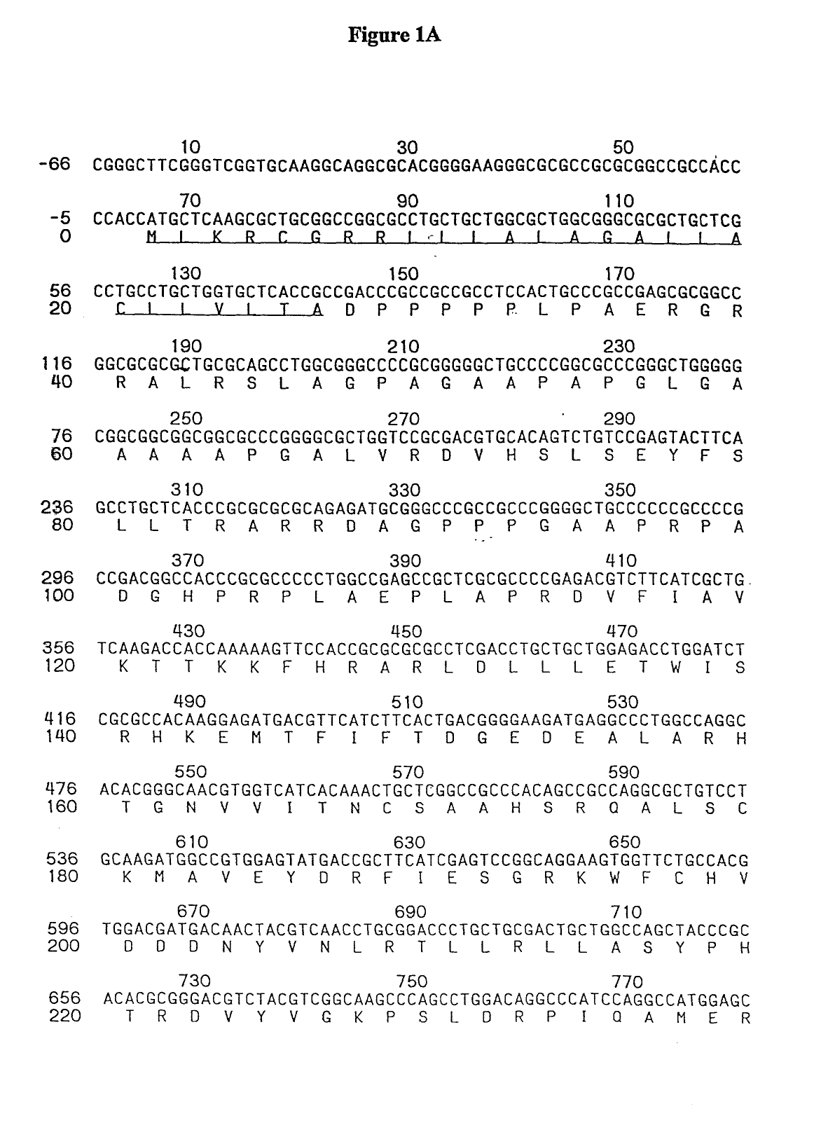

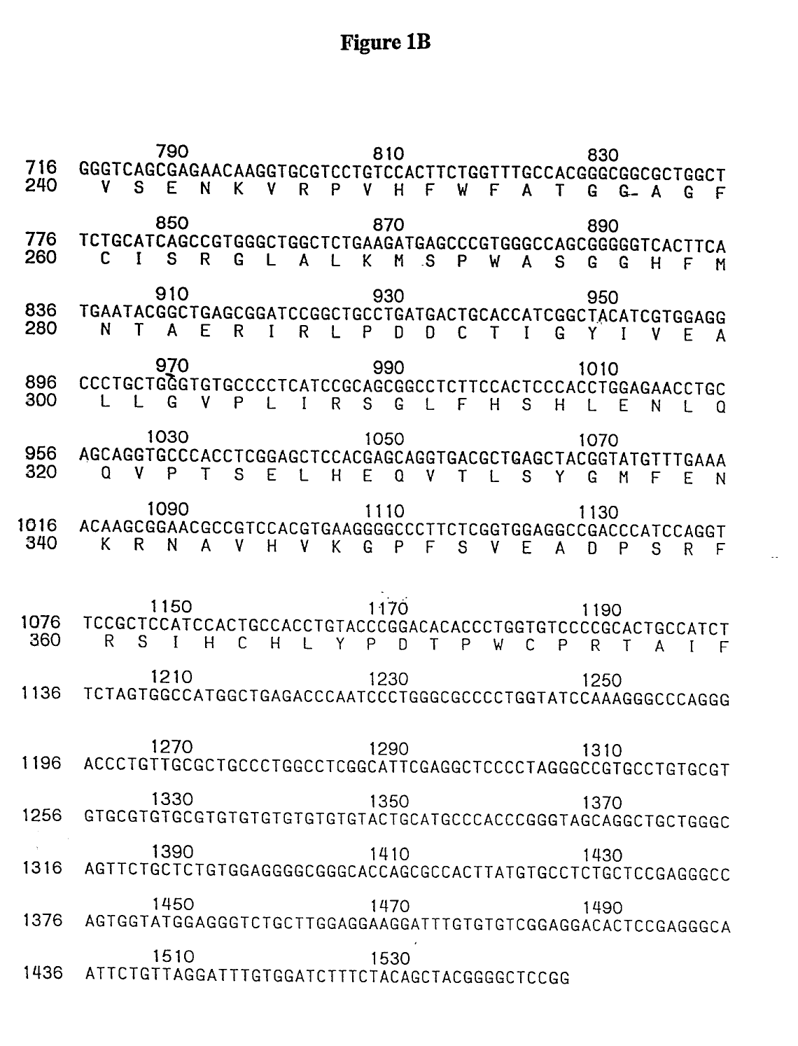

### DETAILED DESCRIPTION

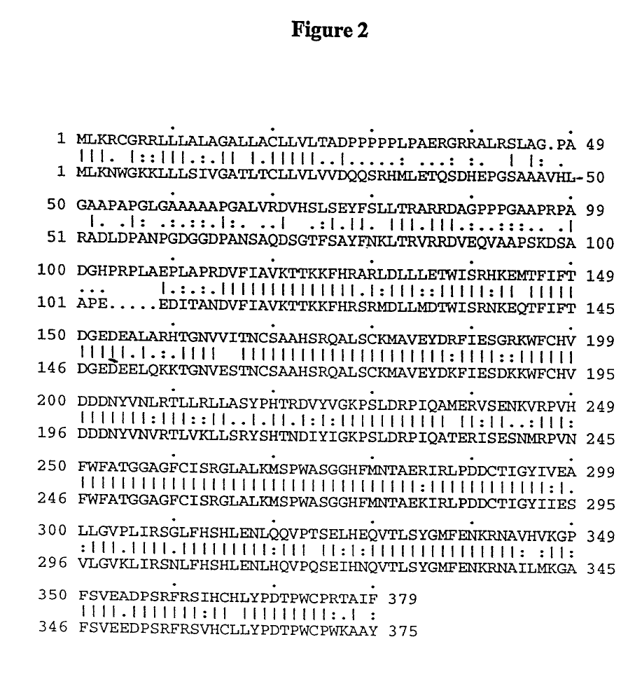

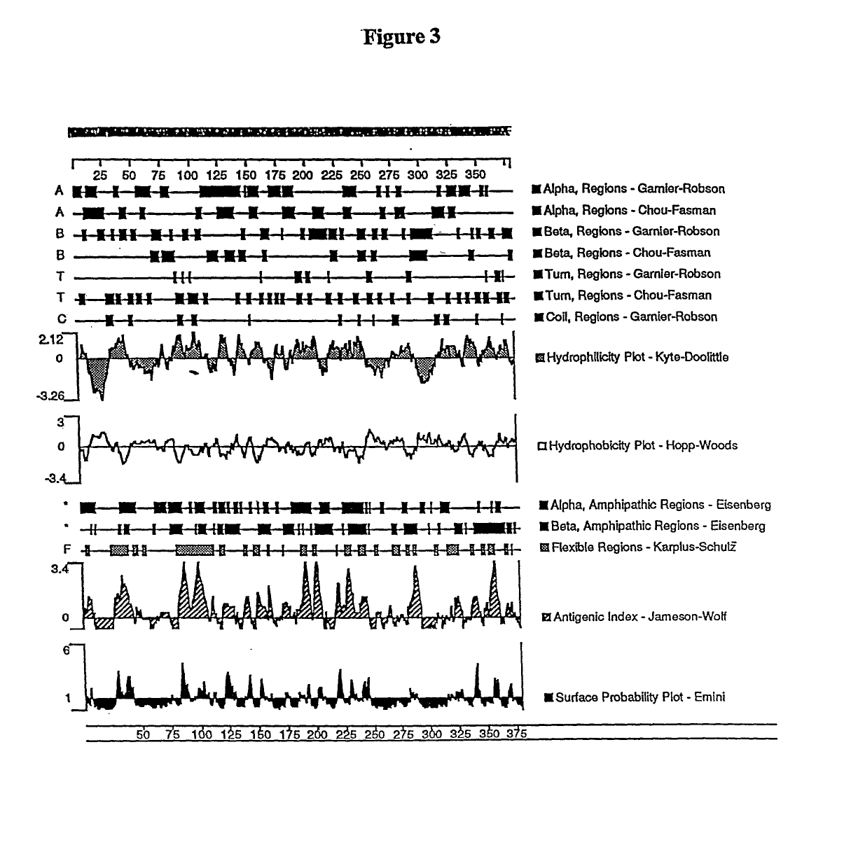

The present invention provides isolated nucleic acid molecules comprising a polynucleotide encoding a HSF polypeptide having the amino acid sequence shown in SEQ ID NO:2, which was determined by sequencing the hlmbp36 cDNA clone. The HSF protein of the present invention shares sequence homology with the Xenopus lunatic fringe protein (FIG. 2) (SEQ ID NO:3).

The hlmbp36 cDNA clone, which encodes the HSF protein, including amino acid residues −26 to 353 in SEQ ID NO:2, was deposited on Sep. 23, 1996 at the American Type Culture Collection, 12301 Park Lawn Drive, Rockville, Md. 20852, and given accession number 97731. The HSF sequence is contained between the EcoR I and Xho I restriction sites in the polylinker of the pBluescript SK(−) plasmid (Stratagene, LaJolla, Calif.).

Nucleic Acid Molecules

Unless otherwise indicated, all nucleotide sequences determined by sequencing a DNA molecule herein were determined using an automated DNA sequencer (such as the Model 373 from Applied Biosystems, Inc.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

Using the information provided herein, such as the nucleotide sequence in SEQ ID NO:1, a nucleic acid molecule of the present invention encoding a HSF polypeptide may be obtained using standard cloning and screening procedures, such as those for cloning cDNAs using mRNA as starting material. Illustrative of the invention, the nucleic acid molecule described in SEQ ID NO:1 was discovered in a cDNA library derived from human breast lymph node. The determined nucleotide sequence of the HSF cDNA of SEQ ID NO:1 contains an open reading frame encoding a protein of about 379 amino acid residues, with a predicted leader sequence of about 26 amino acid residues, and a deduced molecular weight of about 38 kDa. The amino acid sequence of the predicted mature HSF is from about amino acid residue 1 to about 353 (SEQ ID NO:2). The HSF protein shown in SEQ ID NO:2 is about 66.31% identical and about 78.88% similar to the Xenopus lunatic fringe (lfng) protein (FIG. 2; SEQ ID NO:3).

The protein of the present invention is a secreted protein, similar to a family of proteins which include the homologous fng protein of Drosophila, and the lunatic fringe (lFng) and radical fringe (rFng) genes of Xenopus (Irvine, K. D. and Wieschaus, E., Cell 79:595 (1994); Wu et al., Science 273:355 (1996)). These three genes have all been found to have vertebrate homologs functioning in cellular communication important for embryonic patterning. In particular, this family of proteins has been identified in mesoderm induction. The protein of the present invention is most homologous at the amino acid level to the lfng gene.

The lfng gene affects mesoderm induction, including generation of hematopoietic and muscle cells (Wu et al., Science 273:355 (1996)). The fng gene encodes a molecule that mediates signaling between distinct cell populations (Ivine, K. D. and Wieschaus, E., Cell 79:595-606 (1994)). The fng gene encodes a putatively secreted protein, and it mediates processes that establish the wing margin and promote wing outgrowth without otherwise affecting dorsal-ventral wing cell identity.

As indicated, the present invention also provides the mature form of the HSF protein of the present invention. According to the signal hypothesis, proteins secreted by mammalian cells have a signal or secretory leader sequence which is cleaved from the mature protein once export of the growing protein chain across the rough endoplasmic reticulum has been initiated. Most mammalian cells and even insect cells cleave secreted proteins with the same specificity. However, in some cases, cleavage of a secreted protein is not entirely uniform, which results in two or more mature species on the protein. Further, it has long been known that the cleavage specificity of a secreted protein is ultimately determined by the primary structure of the complete protein, that is, it is inherent in the amino acid sequence of the polypeptide. Therefore, the present invention provides a nucleotide sequence encoding the mature HSF polypeptides having the amino acid sequence encoded by the cDNA clone contained in the host identified as ATCC Deposit No. 97731 and as shown in SEQ ID NO:2. By the mature HSF protein having the amino acid sequence encoded by the cDNA clone contained in the host identified as ATCC Deposit 97731 is meant the mature form(s) of the HSF protein produced by expression in a mammalian cell (e.g., COS cells, as described below) of the complete open reading frame encoded by the human DNA sequence of the clone contained in the vector in the deposited host. As indicated below, the mature HSF having the amino acid sequence encoded by the cDNA clone contained in ATCC Deposit No. 97731 may or may not differ from the predicted “mature” HSF protein shown in SEQ ID NO:2 (amino acids from about 1 to about 353), depending on the accuracy of the predicted cleavage site based on computer analysis.

Methods for predicting whether a protein has a secretory leader as well as the cleavage point for that leader sequence are available. For instance, the methods of McGeoch (Virus Res. 3:271-286 (1985)) and von Heinje Nucleic Acids Res. 14:4683-4690 (1986)) can be used. The accuracy of predicting the cleavage points of known mammalian secretory proteins for each of these methods is in the range of 75-80%. von Heinje, supra. However, the two methods do not always produce the same predicted cleavage point(s) for a given protein.

In the present case, the predicted amino acid sequence of the complete hCRY2 polypeptide of the present invention was analyzed by a computer program (“PSORT”) (K. Nakai and M. Kanehisa, Genomics 14:897-911 (1992)), which is an expert system for predicting the cellular location of a protein based on the amino acid sequence. As part of this computational prediction of localization, the methods of McGeoch and von Heinje are incorporated. The analysis by the PSORT program predicted the cleavage sites between amino acids −1 and 1 in SEQ ID NO:2. Thus, the leader sequence for the HSF protein is predicted to consist of amino acid residues −26 to −1 in SEQ ID NO:2, while the mature HSF protein is predicted to consist of amino acids residues 1-353 in SEQ ID NO:2.

As one of ordinary skill would appreciate, due to the possibilities of sequencing errors discussed above, as well as the variability of cleavage sites for leaders in different known proteins, the predicted HSF polypeptide encoded by the deposited cDNA comprises about 379 amino acids, but may be anywhere in the range of 370 to about 390 amino acids; and the predicted leader sequence of this protein is about 26 amino acids, but may be anywhere in the range of about 20 to about 32 amino acids.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) shown in SEQ ID NO:1; DNA molecules comprising the coding sequence for the mature HSF protein (last 353 amino acids of SEQ ID NO:2); and DNA molecules which comprise a sequence substantially different from those described above but which, due to the degeneracy of the genetic code, still encode the HSF protein. Of course, the genetic code is well known in the art. Thus, it would be routine for one skilled in the art to generate such degenerate variants.

In another aspect, the invention provides isolated nucleic acid molecules encoding the HSF polypeptide having an amino acid sequence encoded by the cDNA set forth in SEQ ID NO:1 and by the clone contained in the plasmid deposited as ATCC Deposit No. 97731 on Sep. 23, 1996. In further embodiments, this nucleic acid molecule will encode the mature polypeptide or the full-length polypeptide lacking the N-terminal methionine. The invention further provides an isolated nucleic acid molecule having the nucleotide sequence shown in SEQ ID NO:1 or the nucleotide sequence of the HSF cDNA contained in the above-described deposited clone, or a nucleic acid molecule having a sequence complementary to one of the above sequences. Such isolated molecules, particularly DNA molecules, are useful as probes for gene mapping, by in situ hybridization with chromosomes, and for detecting expression of the HSF gene in human tissue, for instance, by Northern blot analysis.

The present invention is further directed to fragments of the isolated nucleic acid molecules described herein. By a fragment of an isolated nucleic acid molecule having the nucleotide sequence of the deposited cDNA or the nucleotide sequence shown in SEQ ID NO:1 is intended fragments at least about 15 nt, and more preferably at least about 20 nt, still more preferably at least about 30 nt, and even more preferably, at least about 40 nt in length which are useful as diagnostic probes and primers as discussed herein. Of course, larger fragments 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500 or nt in length are also useful according to the present invention as are fragments corresponding to most, if not all, of the nucleotide sequence of the deposited cDNA or as shown in SEQ ID NO:1. By a fragment at least 20 nt in length, for example, is intended fragments which include 20 or more contiguous bases from the nucleotide sequence of the deposited cDNA or the nucleotide sequence as shown in SEQ ID NO:1.

Preferred nucleic acid fragments of the present invention also include nucleic acid molecules encoding epitope-bearing portions of the HSF protein. In particular, such nucleic acid fragments of the present invention include nucleic acid molecules encoding: a polypeptide comprising amino acid residues from about −26 to about −17 in SEQ ID NO:2; a polypeptide comprising amino acid residues from about 1 to about 26 in SEQ ID NO:2; a polypeptide comprising amino acid residues from about 56 to about 90 in SEQ ID NO2; a polypeptide comprising amino acid residues from about 94 to about 106 in SEQ ID NO:2; a polypeptide comprising amino acid residues from about 112 to about 137 in SEQ ID NO:2; a polypeptide comprising amino acid residues from about 146 to about 181 in SEQ ID NO:2; a polypeptide comprising amino acid residues from about 191 to about 222 in SEQ ID NO:2; a polypeptide comprising amino acid residues from about 257 to about 266 in SEQ ID NO:2; a polypeptide comprising amino acid residues from about 293 to about 304 in SEQ ID NO:2; and a polypeptide comprising amino acid residues from about 311 to about 351 in SEQ ID NO:2. It is believed that the above polypeptide fragments are antigenic regions of the HSF protein. Methods for determining other such epitope-bearing portions of the HSF protein are described in detail below.

In addition, the present inventors have identified the following cDNA clones related to extensive portions of SEQ ID NO:1: HJPAS16R (SEQ ID NO:11); and HNHFN35R (SEQ ID NO:12).

The following public ESTs, which relate to portions of SEQ ID NO:1, have also been identified: GenBank Accession No. AA183096 (SEQ ID NO:13); GenBank Accession No. R56561 (SEQ ID NO:14); and GenBank Accession No. AA138083 (SEQ ID NO:15).

In another aspect, the invention provides an isolated nucleic acid molecule comprising a polynucleotide which hybridizes under stringent hybridization conditions to a portion of the polynucleotide in a nucleic acid molecule of the invention described above, for instance, the cDNA clone contained in ATCC Deposit 97731. By “stringent hybridization conditions” is intended overnight incubation at 42° C. in a solution comprising: 50% formamide, 5× SSC (150 mM NaCl, 15 mM trisodium citrate), 50 mM sodium phosphate (pH 7.6), 5× Denhardt's solution, 10% dextran sulfate, and 20 g/ml denatured, sheared salmon sperm DNA, followed by washing the filters in 0.1× SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 nt of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotide (e.g., the deposited cDNA or the nucleotide sequence as shown in SEQ ID NO:1). Of course, a polynucleotide which hybridizes only to a poly A sequence (such as the 3′ terminal poly(A) tract of the HSF cDNA shown in SEQ ID NO:1), or to a complementary stretch of T (or U) resides, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention which encode a HSF polypeptide may include, but are not limited to those encoding the amino acid sequence of the mature polypeptide, by itself; the coding sequence for the mature polypeptide and additional sequences, such as those encoding the about 26 amino acid leader or secretory sequence, such as a pre-, or pro- or prepro-protein sequence; the coding sequence of the mature polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities. Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (Qiagen, Inc.), among others, many of which are commercially available. As described in Gentz et al., Proc. Natl. Acad Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37: 767 (1984). As discussed below, other such fusion proteins include the HSF fused to Fc at the N- or C-terminus.

The present invention further relates to variants of the nucleic acid molecules of the present invention, which encode portions, analogs or derivatives of the HSF protein. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques.

Such variants include those produced by nucleotide substitutions, deletions or additions, which may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the HSF protein or portions thereof. Also especially preferred in this regard are conservative substitutions.

Further embodiments of the invention include isolated nucleic acid molecules comprising a polynucleotide having a nucleotide sequence at least 95% identical, and more preferably at least 96%, 97%, 98% or 99% identical to (a) a nucleotide sequence encoding the full-length HSF polypeptide having the complete amino acid sequence SEQ ID NO:2, including the leader sequence (amino acid residues about −26 to about 353 in SEQ ID NO:2); (b) a nucleotide sequence encoding the polypeptide having the complete amino acid sequence in SEQ ID NO:2 except for the N-terminal methionine (amino acids residues about −25 to about 353 in SEQ ID NO:2); (c) a nucleotide sequence encoding the polypeptide having the amino acid sequence at positions from about 1 to about 353 in SEQ ID NO:2; (d) a nucleotide sequence encoding the HSF polypeptide having the complete amino acid sequence encoded by the cDNA clone contained in ATCC Deposit No. 97731; (e) a nucleotide sequence encoding the mature HSF polypeptide having the amino acid sequence encoded by the cDNA clone contained in ATCC Deposit No. 97731; or (f) a nucleotide sequence complementary to any of the nucleotide sequences in (a), (b), (c), (d), or (e).

By a polynucleotide having a nucleotide sequence at least, for example, 95% “identical” to a reference nucleotide sequence encoding a HSF polypeptide is intended that the nucleotide sequence of the polynucleotide is identical to the reference sequence except that the polynucleotide sequence may include up to five point mutations per each 100 nucleotides of the reference nucleotide sequence encoding the HSF polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 95%, 96%, 97%, 98% or 99% identical to, for instance, the nucleotide sequence shown in SEQ ID NO:1 or to the nucleotide sequence of the deposited cDNA clone can be determined conventionally using known computer programs such as the Bestfit program (Wisconsin Sequence Analysis Package, Version 8 for Unix, Genetics Computer Group, University Research Park, 575 Science Drive, Madison, Wis. 53711. Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 95%, 96%, 97%, 98% or 99% identical to the nucleic acid sequence shown in SEQ ID NO:1 or to the nucleic acid sequence of the deposited cDNA, irrespective of whether they encode a polypeptide having HSF activity. This is because even where a particular nucleic acid molecule does not encode a polypeptide having HSF activity, one of skill in the art would still know how to use the nucleic acid molecule, for instance, as a hybridization probe or a polymerase chain reaction (PCR) primer. Uses of the nucleic acid molecules of the present invention that do not encode a polypeptide having HSF activity include, inter alia, (1) isolating the HSF gene or allelic variants thereof in a cDNA library; (2) in situ hybridization (e.g., “FISH”) to metaphase chromosomal spreads to provide precise chromosomal location of the HSF gene, as described in Verma et al., Human Chromosomes: A Manual of Basic Techniques, Pergamon Press, New York (1988); and (3) Northern Blot analysis for detecting HSF mRNA expression in specific tissues.

Preferred, however, are nucleic acid molecules having sequences at least 95%, 96%, 97%, 98% or 99% identical to the nucleic acid sequence shown in SEQ ID NO:1 or to the nucleic acid sequence of the deposited cDNA which do, in fact, encode a polypeptide having HSF protein activity. By “a polypeptide having HSF activity” is intended polypeptides exhibiting activity similar, but not necessarily identical, to an activity of the HSF protein of the invention (either the full-length protein or, preferably, the mature protein), as measured in a particular biological assay. For example, HSF protein activity can be measured using the in vitro colony forming assay as described in Youn et al., The Journal of Immunology 155:2661-2667 (1995). Briefly, the assay involves collecting human or murine bone marrow cells and plating the same on agar, adding one or more growth factors and either (1) transfected host cell-supernatant containing HSF protein (or a candidate polypeptide) or (2) nontransfected host cell-supernatant control, and measuring the effect on colony formation by murine and human CFU-granulocyte-macrophages (CFU-GM), by human burst-forming unit-erythroid (BFU-E), or by human CFU granulocyte-erythroid-macrophage-megakaryocyte (CFU-GEMM).

Of course, due to the degeneracy of the genetic code, one of ordinary skill in the art will immediately recognize that a large number of the nucleic acid molecules having a sequence at least 95%, 96%, 97%, 98%, or 99% identical to the nucleic acid sequence of the deposited cDNA or the nucleic acid sequence shown in SEQ ID NO:1 will encode a polypeptide “having HSF protein activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having HSF protein activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that proteins are surprisingly tolerant of amino acid substitutions.

Vectors and Host Cells

The present invention also relates to vectors which include the isolated DNA molecules of the present invention, host cells which are genetically engineered with the recombinant vectors, and the production of HSF polypeptides or fragments thereof by recombinant techniques.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art.

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize proteins. For example, EP-A-O 464 533 (Canadian counterpart 2045869) discloses fusion proteins comprising various portions of constant region of immunoglobin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as, hIL5- has been fused with Fc portions for the purpose of high-throughput screening assays to identify antagonists of hIL-5. See, D. Bennett et al., Journal of Molecular Recognition, Vol. 8 52-58 (1995) and K. Johanson et al., The Journal of Biological Chemistry, Vol. 270, No. 16, pp 9459-9471 (1995).

The HSF protein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

HSF Polypeptides and Fragments

The invention further provides an isolated HSF polypeptide having the amino acid sequence encoded by the deposited cDNA, or the amino acid sequence in SEQ ID NO:2, or a peptide or polypeptide comprising a portion of the above polypeptides.

It will be recognized in the art that some amino acid sequences of the HSF polypeptide can be varied without significant effect of the structure or function of the protein. If such differences in sequence are contemplated, it should be remembered that there will be critical areas on the protein which determine activity.

Thus, the invention further includes variations of the HSF polypeptide which show substantial HSF polypeptide activity or which include regions of HSF protein such as the protein portions discussed below. Such mutants include deletions, insertions, inversions, repeats, and type substitutions. As indicated above, guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Thus, the fragment, derivative or analog of the polypeptide of SEQ ID NO:2, or that encoded by the deposited cDNA, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence which is employed for purification of the mature polypeptide or a pro-protein sequence. Such fragments, derivatives and analogs are deemed to be within the scope of those skilled in the art from the teachings herein.

Of particular interest are substitutions of charged amino acids with another charged amino acid and with neutral or negatively charged amino acids. The latter results in proteins with reduced positive charge to improve the characteristics of the HSF protein. The prevention of aggregation is highly desirable. Aggregation of proteins not only results in a loss of activity but can also be problematic when preparing pharmaceutical formulations, because they can be immunogenic (Pinckard et al., Clin. Exp. Immunol. 2:331-340 (1967); Robbins et al., Diabetes 36:838-845 (1987); Cleland et al,. Crit. Rev. Therapeutic Drug Carrier Systems 10:307-377 (1993)).

The replacement of amino acids can also change the selectivity of binding to cell surface receptors. Ostade et al., Nature 361:266-268 (1993) describes certain mutations resulting in selective binding of TNF-α to only one of the two known types of TNF receptors. Thus, the HSF of the present invention may include one or more amino acid substitutions, deletions or additions, either from natural mutations or human manipulation.

As indicated, changes are preferably of a minor nature, such as conservative amino acid substitutions that do not significantly affect the folding or activity of the protein (see Table 1).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Of course, the number of amino acid substitutions a skilled artisan would make depends on many factors, including those described above. Generally speaking, the number of amino acid substitutions for any given HSF polypeptide will not be more than 50, 40, 30, 20, 10, 5, or 3.

Amino acids in the HSF protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro proliferative activity.

Sites that are critical for ligand-receptor binding can also be determined by structural analysis such as crystallization, nuclear magnetic resonance or photoaffinity labeling (Smith et al., J. Mol. Biol. 224:899-904 (1992); and de Vos et al. Science 255:306-312 (1992)).

The polypeptides of the present invention are preferably provided in an isolated form. By “isolated polypeptide” is intended a polypeptide removed from its native environment. Thus, a polypeptide produced or contained in a recombinant host cell is considered “isolated” for the purposes of the present invention. Also intended as “isolated” is a polypeptide that has been purified, partially or substantially, from a recombinant host or from a native source. For example, a recombinantly produced version of the HSF receptor can be substantially purified by the one-step method described in Smith and Johnson, Gene 67:31-40 (1988).

The polypeptides of the present invention also include the complete polypeptide encoded by the deposited cDNA; the mature polypeptide encoded by the deposited cDNA; amino acid residues from about −26 to about 353 of SEQ ID NO:2; amino acid residues from about −25 to about 353 of SEQ ID NO:2; and amino acid residues from about 1 to about 353 of SEQ ID NO:2, as well as polypeptides which are at least 95% identical, and more preferably at least 96%, 97%, 98% or 99% identical to the polypeptide encoded by the deposited cDNA, to the polypeptide of SEQ ID NO:2, and also include portions of such polypeptides with at least 30 amino acids and more preferably at least 50 amino acids.

By a polypeptide having an amino acid sequence at least, for example, 95% “identical” to a reference amino acid sequence of a HSF polypeptide is intended that the amino acid sequence of the polypeptide is identical to the reference sequence except that the polypeptide sequence may include up to five amino acid alterations per each 100 amino acids of the reference amino acid of the HSF polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 95%, 96%, 97%, 98% or 99% identical to, for instance, the amino acid sequence shown in SEQ ID NO:2 or to the amino acid sequence encoded by deposited cDNA clone can be determined conventionally using known computer programs such the Bestfit program (Wisconsin Sequence Analysis Package, Version 8 for Unix, Genetics Computer Group, University Research Park, 575 Science Drive, Madison, Wis. 53711. When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The polypeptide of the present invention could be used as a molecular weight marker on SDS-PAGE gels or on molecular sieve gel filtration columns using methods well known to those of skill in the art.

In another aspect, the invention provides a peptide or polypeptide comprising an epitope-bearing portion of a polypeptide of the invention. The epitope of this polypeptide portion is an immunogenic or antigenic epitope of a polypeptide described herein. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Learner, R. A., Antibodies that react with predetermined sites on proteins, Science 219:660-666 (1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals.

Antigenic epitope-bearing peptides and polypeptides of the invention are therefore useful to raise antibodies, including monoclonal antibodies, that bind specifically to a polypeptide of the invention. See, for instance, Wilson et al., Cell 37:767-778 (1984) at 777.

Antigenic epitope-bearing peptides and polypeptides of the invention preferably contain a sequence of at least seven, more preferably at least nine and most preferably between about 15 to about 30 amino acids contained within the amino acid sequence of a polypeptide of the invention.

Non-limiting examples of antigenic polypeptides or peptides that can be used to generate HSF-specific antibodies include: a polypeptide comprising amino acid residues from about −26 to about −17 in SEQ ID NO:2; a polypeptide comprising amino acid residues from about 1 to about 26 in SEQ ID NO:2; a polypeptide comprising amino acid residues from about 56 to about 90 in SEQ ID NO:2; a polypeptide comprising amino acid residues from about 94 to about 106 in SEQ ID NO:2; a polypeptide comprising amino acid residues from about 112 to about 137 in SEQ ID NO:2; a polypeptide comprising amino acid residues from about 146 to about 181 in SEQ ID NO:2; a polypeptide comprising amino acid residues from about 191 to about 222 in SEQ ID NO:2; a polypeptide comprising amino acid residues from about 257 to about 266 in SEQ ID NO:2; a polypeptide comprising amino acid residues from about 293 to about 304 in SEQ ID NO:2; and a polypeptide comprising amino acid residues from and about 311 to about 351 in SEQ ID NO:2. As indicated above, it is believed that the above polypeptide fragments are antigenic regions of the HSF protein.

The epitope-bearing peptides and polypeptides of the invention may be produced by any conventional means. See, for example, Houghten, R. A., General method for the rapid solid-phase synthesis of large numbers of peptides: specificity of antigen-antibody interaction at the level of individual amino acids. Proc. Natl. Acad. Sci. USA 82:5131-5135 (1985). This “Simultaneous Multiple Peptide Synthesis (SMPS)” process is further described in U.S. Pat. No. 4,631,211 to Houghten et al. (1986).

As one of skill in the art will appreciate, HSF polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric HSF protein or protein fragment alone (Fountoulakis et al., J. Biochem. 270:3958-3964 (1995)).

Diagnostic and Prognostic Applications ofHSF

It is believed that mammals with certain hematopoietic disorders express significantly altered levels of the HSF protein and mRNA encoding the HSF protein when compared to a corresponding “standard” mammal, i.e., a mammal of the same species not having the hematopoietic disorder. Hematopoietic tissues or cells from which samples can be obtained include the spleen, thymus, bone marrow, erythrocytes, neutrophils, granulocytes, monocytes, eosinophils, mast cells megakaryocytes, T-cells, B-cells, natural killer cells, and macrophages. Further, it is believed that significantly altered levels of the HSF protein can be detected in certain body fluids (e.g., bone marrow, lymph fluid, blood, sera, plasma, saliva, urine, synovial fluid and spinal fluid) from mammals with the hematopoietic disorder when compared to sera from mammals of the same species not having the hematopoietic disorder. Thus, the invention provides a diagnostic method useful during the diagnosis of hematopoietic disorders, which involves assaying the expression level of the gene encoding the HSF protein in mammalian cells or body fluid and comparing the gene expression level with a standard HSF gene expression level, whereby a decrease in the gene expression level over the standard is indicative of certain hematopoietic disorders. Preferred mammals include monkeys, apes, cats, dogs, cows, pigs, horses, rabbits and humans. Particularly preferred are humans.

Hematopoietic disorders which can be diagnosed include but are not limited to leukemias (e.g., acute myeloid leukemia (promyelocytic, monocytic) acute lymphoblastic leukemia, common acute lymphoblastic leukemia, pre-B acute lymphoblastic leukemia, B-cell chronic lymphocytic leukemia, B-cell prolymphocytic leukemia, hairy cell leukemia, T-cell chronic lymphocytic leukemia, T-cell prolymphocytic leukemia, chronic myeloid leukemia, Sezary syndrome, multiple myeloma), lymphoma (e.g., malignant lymphomas, sarcoma, extranodal lymphomas, hitiocytic lymphoma, malignant histiocytosis), Hodgkin's disease, non-Hodgkin's lymphomas (e.g., T-cell non-Hodgkin's lymphoma, B-cell non-Hodgkin's lymphoma, lymphocytic non-Hodgkin's lymphoma, follicle center cell non-Hodgkin's lymphoma, immunoblastic non-Hodgkin's lymphoma, immunocytoma, lymphoblastic non-Hodgkin's lymphoma, and multiple myeloma). See Lymphoproliferative Diseases, Jones, D. B. et al., Eds., Kluwer Academic Publishers (1990); The Lymphoid Leukemias, Catovsky, D. et al., Butterworths & Co. (1990); and Sachs, L, Cancer 65:2196 (1990).

Where a diagnosis of a hematopoietic disorder has already been made according to conventional methods, the present invention is useful as a prognostic indicator, whereby patients exhibiting decreased HSF gene expression will experience a worse clinical outcome relative to patients expressing the gene at a lower level. Hematopoietic disorders for which the prognosis can be determined includes but is not limited to the hematopoietic disorders listed above.

By “assaying the expression level of the gene encoding the HSF protein” is intended qualitatively or quantitatively measuring or estimating the level of the HSF protein or the level of the mRNA encoding the HSF protein in a first biological sample either directly (e.g., by determining or estimating absolute protein level or mRNA level) or relatively (e.g., by comparing to the HSF protein level or mRNA level in a second biological sample).

Preferably, the HSF protein level or mRNA level in the first biological sample is measured or estimated and compared to a standard HSF protein level or mRNA level, the standard being taken from a second biological sample obtained from an individual not having the hematopoietic disorder. As will be appreciated in the art, once a standard HSF protein level or mRNA level is known, it can be used repeatedly as a standard for comparison.

By “biological sample” is intended any biological sample obtained from an individual, cell line, tissue culture, or other source which contains HSF protein or mRNA. Biological samples include mammalian body fluids (such as bone marrow, lymph fluid, blood, sera, plasma, urine, synovial fluid and spinal fluid) which contain secreted mature HSF protein, and thymus, bone marrow, lymph node, ovarian, prostate, heart, placenta, pancreas, liver, spleen, lung, breast, erythrocytes, neutrophils, granulocytes, monocytes, eosinophils, mast cells megakaryocytes, T-cells, B-cells, natural killer cells, macrophages, and umbilical tissue.

Total cellular RNA can be isolated from a biological sample using the single-step guanidinium-thiocyanate-phenol-chloroform method described in Chomczynski and Sacchi, Anal. Biochem. 162:156-159 (1987). Levels of mRNA encoding the HSF protein are then assayed using any appropriate method. These include Northern blot analysis (Harada et al., Cell 63:303-312 (1990)), S1 nuclease mapping (Fujita et al., Cell 49:357-367 (1987)), the polymerase chain reaction (PCR), reverse transcription in combination with the polymerase chain reaction (RT-PCR) (Makino et al., Technique 2:295-301 (1990)), and reverse transcription in combination with the ligase chain reaction (RT-LCR).

Assaying HSF protein levels in a biological sample can occur using antibody-based techniques. For example, HSF protein expression in tissues can be studied with classical immunohistological methods (Jalkanen, M., et al., J. Cell. Biol. 101:976-985 (1985); Jalkanen, M., et al., J. Cell. Biol. 105:3087-3096 (1987)).

Other antibody-based methods useful for detecting HSF protein gene expression include immunoassays, such as the enzyme linked immunosorbent assay (ELISA) and the radioimmunoassay (RIA). Suitable labels are known in the art and include enzyme labels, such as glucose oxidase, and radioisotopes, such as iodine (¹²⁵I, ¹²¹I), carbon (¹⁴C), sulphur (³⁵S), tritium (³H), indium (¹¹²In), and technetium (^{99m}Tc), and fluorescent labels, such as fluorescein and rhodamine, and blotin.

HSF Protein and Antibody Therapy

A large number of disease conditions are associated with modifications of the hematopoietic signaling system (Sachs, L, Cancer 65:2196 (1990)). Examples of such disease conditions include, but are not limited to leukemias (e.g., acute myeloid leukemia (promyelocytic, monocytic) acute lymphoblastic leukemia, common acute lymphoblastic leukemia, pre-B acute lymphoblastic leukemia, B-cell chronic lymphocytic leukemia, B-cell prolymphocytic leukemia, hairy cell leukemia, T-cell chronic lymphocytic leukemia, T-cell prolymphocytic leukemia, chronic myeloid leukemia, Sezary syndrome, multiple myeloma), lymphoma (e.g., malignant lymphomas, sarcoma, extranodal lymphomas, histiocytic lymphoma, malignant histiocytosis), Hodgkin's disease, non-Hodgkin's lymphomas (e.g., T-cell non-Hodgkin's lymphoma, B-cell non-Hodgkin's lymphoma, lymphocytic non-Hodgkin's lymphoma, follicle center cell non-Hodgkin's lymphoma, immunoblastic non-Hodgkin's lymphoma, immunocytoma, lymphoblastic non-Hodgkin's lymphoma, and multiple myeloma). See Lymphoproliferative Diseases, Jones, D. B. et al., Eds., Kluwer Academic Publishers (1990); The Lymphoid Leukemias, Catovsky, D. et al., Butterworths & Co. (1990); and Sachs, L, Cancer 65:2196 (1990). Because of the role of the HSF system in these disease states, activation of the hematopoietic system by HSF should provide therapeutic benefits to an individual suffering from one (or more) of these physiologic or pathologic diseases.

Given the hematopoietic activities modulated by HSF, it is readily apparent that a substantially altered level of expression of HSF in an individual, compared to the standard or “normal” level, may produce pathological conditions such as those described above in relation to diagnosis. It will also be appreciated by one of ordinary skill that, since the HSF protein of the invention is translated with a leader peptide suitable for secretion of the mature protein from the cells which express HSF, when HSF protein (particularly the mature form) is added from an exogenous source to cells, tissues or the body of an individual, the protein will exert its modulating activities on any of its target cells of that individual. Therefore, it will be appreciated that conditions caused by a decrease in the standard or normal level of HSF activity in an individual, can be treated by administration of HSF protein. Thus, the invention also provides a method of treatment of an individual in need of an increased level of activity comprising administering to such an individual a pharmaceutical composition comprising an amount of an isolated HSF polypeptide of the invention, particularly a mature form of the HSF protein of the invention, effective to increase the HSF activity level in such an individual.

Moreover, it is believed that the HSF protein can be used to modulate the development of hematopoietic stem cells in vivo or ex vivo. For in vivo applications, the HSF protein or a fragment thereof can be administered to a mammal. Stem cell expansion can be measured using techniques familiar to those of ordinary skill in the art. For example, bone marrow can be aspirated and changes in stem cell levels can be quantified using fluorescence activated cell sorting (FACS).

In addition, stem cells obtained from umbilical cord blood can be cultured, expanded, and then caused to differentiate into various types of hematopoietic cells ex vivo. After expansion and/or differentiation, the cells can be transplanted into human or animal subjects in need thereof. To facilitate stem cell expansion and/or differentiation, various growth factors (e.g., cytokines) are added to the culture. Techniques for culturing, expanding stem cells and for causing differentiating cord blood stem cells to differentiate into other hematopoietic cell types are well known to those of ordinary skill in the art. For example, see Almici, C. et al., Acta Haematol 95: 171-175 (1996); Almici, C. et al., Haematologica 80:473-479 (1995); Hatzfeld, J. et al., Blood Cells 20:430-434 (1994); Risdon, G. et al., Blood Cells 20:566-570 (1994); Van Epps, D. E. et al., Blood Cells 20:411-423 (1994); and Urashima, M. et al., Acta Paediatr. Japan 36:649-655 (1994). Cultured stem cells can be treated with HSF protein alone or with HSF protein and other growth factors.

It is believed that the HSF protein modulates (either increases or decreases) the response of activated neutrophils in acute inflammatory conditions.

Subjects suffering from illnesses which are due, at least in part, to an abnormally high level of HSF protein (e.g., hematopoietic disease conditions listed above, acute inflammation, or chronic inflammation) will benefit from anti-HSF antibody therapy. Antibody-based therapies involve administering an anti-HSF antibody to a mammalian, preferably human, patient for treating one or more of the above-described disorders. Methods for producing anti-HSF polyclonal and monoclonal antibodies are described in detail above. Such antibodies may be provided in pharmaceutically acceptable compositions as known in the art or as described herein.

A summary of the ways in which the antibodies of the present invention may be used therapeutically includes administering HSF locally or systemically in the body or by direct cytotoxicity of the antibody, e.g., as mediated by complement or by effector cells. Some of these approaches are described in more detail below. Armed with the teachings provided herein, one of ordinary skill in the art will know how to use the antibodies of the present invention for diagnostic, monitoring or therapeutic purposes without undue experimentation.

The pharmaceutical compositions of the present invention may be administered by any means that achieve their intended purpose. Amounts and regimens for the administration of antibodies, their fragments or derivatives can be determined readily by those with ordinary skill in the clinical art of treating HSF-related disease.

For example, administration may be by parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

Compositions within the scope of this invention include all compositions wherein the antibody, fragment or derivative is contained in an amount effective to achieve its intended purpose. While individual needs vary, determination of optimal ranges of effective amounts of each component is within the skill of the art. The effective dose is a function of the individual chimeric or monoclonal antibody, the presence and nature of a conjugated therapeutic agent (see below), the patient and his or her clinical status, and can vary from about 10 μg/kg body weight to about 5000 mg/kg body weight. The preferred dosages comprise 0.1 to 500 mg/kg body weight.

In addition to pharmacologically active compounds, the new pharmaceutical compositions may contain suitable pharmaceutically acceptable carriers comprising excipients and auxiliaries which facilitate process of the active compounds into preparations which can be used pharmaceutically. Preferably, the preparations contain from about 0.01 to 99 percent, preferably from about 20 to 75 percent of active compound(s), together with the excipient.

Similarly, preparations of an HSF antibody or fragment of the present invention for parenteral administration, such as in detectably labeled form for imaging or in a free or conjugated form for therapy, include sterile aqueous or non-aqueous solutions, suspensions, and emulsions. Examples of non-aqueous solvents are propylene glycol, polyethylene glycol, vegetable oil such as olive oil, and injectable organic esters such as ethyl oleate. Aqueous carriers include water, alcoholic/aqueous solutions, emulsions or suspensions, including saline and buffered media, parenteral vehicles including sodium chloride solution, Ringer's dextrose, dextrose and sodium chloride, lactated Ringer's, or fixed oils. Intravenous vehicles include fluid and nutrient replenishers, such as those based on Ringer's dextrose, and the like. Preservatives and other additives may also be present, such as, for example, antimicrobials, anti-oxidants, chelating agents, and inert gases and the like. See, generally, Remington's Pharmaceutical Science, 18th Ed., Mack Publishing Co., Easton, Pa. (1990).

In particular, the antibodies, fragments and derivatives of the present invention are useful for treating a subject having or developing HSF-related disorders as described herein. Such treatment comprises parenterally administering a single or multiple doses of the antibody, fragment or derivative, or a conjugate thereof.

The antibodies of this invention may be advantageously utilized in combination with other monoclonal or chimeric antibodies, or with lymphokines or hemopoietic growth factors, etc., which serve to increase the number or activity of effector cells which interact with the antibodies.

It is preferred to use high affinity and/or potent in vivo HSF-inhibiting and/or neutralizing antibodies, fragments or regions thereof, for both HSF immunoassays and therapy of HSF related disorders. Such antibodies, fragments, or regions, will preferably have an affinity for human HSF, expressed as Kₐ, of a least 10⁸ M⁻¹, more preferably, at least 10⁹ M⁻¹, such as 5×10⁸ M⁻¹, 8×10⁸ M⁻¹, 2×10⁹ M⁻¹, 4×10⁹ M⁻¹, 6×10⁹ M⁻¹, and 8×10⁹M⁻¹.

One of ordinary skill will appreciate that effective amounts of the HSF polypeptides for treating an individual in need of an increased level of HSF activity (including amounts of HSF polypeptides effective for the conditions discussed above) can be determined empirically for each condition where administration of HSF is indicated.

Modes of administration

It will be appreciated that conditions caused by a decrease in the standard or normal level of HSF activity in an individual, can be treated by administration of HSF protein. Thus, the invention further provides a method of treating an individual in need of an increased level of HSF activity comprising administering to such an individual a pharmaceutical composition comprising an effective amount of an isolated HSF polypeptide of the invention, particularly a mature form of the HSF, effective to increase the HSF activity level in such an individual.

As a general proposition, the total pharmaceutically effective amount of HSF polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the HSF polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed.

Pharmaceutical compositions containing the HSF of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraarticular injection and infusion.

Chromosome Assays

The nucleic acid molecules of the present invention are also valuable for chromosome identification. The sequence is specifically targeted to and can hybridize with a particular location on an individual human chromosome. The mapping of DNAs to chromosomes according to the present invention is an important first step in correlating those sequences with genes associated with disease.

In certain preferred embodiments in this regard, the cDNA herein disclosed is used to clone genomic DNA of a HSF protein gene. This can be accomplished using a variety of well known techniques and libraries, which generally are available commercially. The genomic DNA then is used for in situ chromosome mapping using well known techniques for this purpose.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes.

Fluorescence in situ hybridization (“FISH”) of a cDNA clone to a metaphase chromosomal spread can be used to provide a precise chromosomal location in one step. This technique can be used with probes from the cDNA as short as 50 or 60 bp. For a review of this technique, see Verma et al., Human Chromosomes: A Manual Of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance In Man, available on-line through Johns Hopkins University, Welch Medical Library. The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

Having generally described the invention, the same will be more readily understood by reference to the following examples, which are provided by way of illustration and are not intended as limiting.

### EXAMPLE 1

### Expression and Purification of HSF in E. coli

The bacterial expression vector pQE9 (pD10) is used for bacterial expression in this example. (QIAGEN, Inc., 9259 Eton Avenue, Chatsworth, Calif., 91311). pQE9 encodes ampicillin antibiotic resistance (“Amp^{r}”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., supra, and suitable single restriction enzyme cleavage sites. These elements are arranged such that an inserted DNA fragment encoding a polypeptide expresses that polypeptide with the six His residues (i.e., a “6× His tag”)) covalently linked to the amino terminus of that polypeptide.

The DNA sequence encoding the desired portion of the HSF protein lacking the hydrophobic leader sequence is amplified from the deposited cDNA clone using PCR oligonucleotide primers which anneal to the amino terminal sequences of the desired portion of the HSF protein and to sequences in the deposited construct 3′ to the cDNA coding sequence. Additional nucleotides containing restriction sites to facilitate cloning in the pQE9 vector are added to the 5′ and 3′ primer sequences, respectively.

For cloning the mature protein, the 5′ primer has the sequence 5′-CACCGTCGACCCGCCGCCGCCTCCACTGC-3′ (SEQ ID NO:4), containing the underlined Sal I restriction site followed by 22 nucleotides of the amino terminal coding sequence of the mature HSF sequence in SEQ ID NO:2. One of ordinary skill in the art would appreciate, of course, that the point in the protein coding sequence where the 5′ primer begins may be varied to amplify a DNA segment encoding any desired portion of the complete HSF protein shorter or longer than the mature form. The 3′ primer has the sequence 5′-GGTCT AAGCTTTGGCCATTAGAAGATGGCAGTGCGGG-3′ (SEQ ID NO:5) containing the underlined Hind III restriction site followed by 19 nucleotides reverse and complementary to nucleotides 1186 to 1189 of SEQ ID NO:1.

The amplified HSF DNA fragment and the vector pQE9 are digested with Sal I and Hind III and the digested DNAs are then ligated together. Insertion of the HSF DNA into the restricted pQE9 vector places the HSF protein coding region downstream from the IPTG-inducible promoter and in-frame with an initiating AUG and the six histidine codons.

The ligation mixture is transformed into competent E. coli cells using standard procedures such as those described in Sambrook et al, Molecular Cloning: a Laboratory Manual, 2nd Ed.; Cold Spring Harbor Laboratory Press, Cold Spring Harbor, N.Y. (1989). E. coli strain Ml5/rep4, containing multiple copies of the plasmid pREP4, which expresses the lac repressor and confers kanamycin resistance (“Kan^{r}”), is used in carrying out the illustrative example described herein. This strain, which is only one of many that are suitable for expressing HSF protein, is available commercially from QIAGEN, Inc., supra. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. Isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 34 hours at 4° C. in 6M guanidine-HCl, pH 8. The cell debris is removed by centrifugation, and the supernatant containing the HSF is dialyzed against 50 mM Na-acetate buffer, pH 6, supplemented with 200 mM NaCl. Alternatively, the protein can be successfully refolded by dialyzing it against 500 mM NaCl, 20% glycerol, 25 mM Tris/HCl pH 7.4, containing protease inhibitors. After renaturation the protein can be purified by ion exchange, hydrophobic interaction and size exclusion chromatography. Alternatively, an affinity chromatography step such as an antibody column can be used to obtain pure HSF protein. The purified protein is stored at 4° C. or frozen at −80° C.

### EXAMPLE 2

### Cloning and Expression of HSF Protein in a Baculovirus Expression System

In this illustrative example, the plasmid shuttle vector pA2 is used to insert the cloned DNA encoding the complete protein, including its naturally associated secretary signal (leader) sequence, into a baculovirus to express the mature HSF protein, using standard methods as described in Summers et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by convenient restriction sites such as BamH I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate viable virus that express the cloned polynucleotide.

Many other baculovirus vectors could be used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39.

The cDNA sequence encoding the full length HSF protein in the deposited clone, including the AUG initiation codon and the naturally associated leader sequence shown in (amino acid residues about −26 to about −1 in SEQ ID NO:2), is amplified using PCR oligonucleotide primers corresponding to the 5′ and 3′ sequences of the gene. The 5′ primer has the sequence 5′-GCGTCTAGACCGCCATCATGCTCAAGCGCTGCGG-3′ (SEQ ID NO:6) containing the underlined Xba I restriction enzyme site, an efficient signal for initiation of translation in eukaryotic cells, as described by Kozak, M., J. Mol. Biol. 196:947-950 (1987), followed by 18 bases of the sequence reverse and complementary to nucleotides 65-82 of the sequence shown SEQ ID NO:1. The 3′ primer is the T7 primer for Bluescript and has the sequence 5′-GTAATACGACTCACTATAGGGC-3′ (SEQ ID NO:7).

The amplified fragment is isolated from a 1% agarose gel using a commercially available kit (“Geneclean,” BIO 101 Inc., La Jolla, Calif.). The fragment then is digested with Xba I and Asp718 (the Asp718 site is in the HSF 3′ untranslated region) and again is purified on a 1% agarose gel. This fragment is designated herein “F1.”

The plasmid is digested with the restriction enzymes Xba I and Asp718 and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V1.”

Fragment F1 and the dephosphorylated plasmid VI are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria are identified that contain the plasmid with the human HSF gene using the PCR method, in which one of the primers that is used to amplify the gene and the second primer is from well within the vector so that only those bacterial colonies containing the HSF gene fragment will show amplification of the DNA. The sequence of the cloned fragment is confirmed by DNA sequencing. This plasmid is designated herein pBacHSF.

Five μg of the plasmid pBacHSF are co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84:7413-7417 (1987). 1 μg of BaculoGold™ virus DNA and 5 μg of the plasmid pBacHSF are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is rocked back and forth to mix the newly added solution. The plate is then incubated for 5 hours at 27° C. After 5 hours, the transfection solution is removed from the plate and 1 ml of Grace's insect medium supplemented with 10% fetal calf serum is added. The plate is put back into an incubator and cultivation is continued at 27° C. for four days.

After four days the supernatant is collected and a plaque assay is performed, as described by Summers and Smith, supra An agarose gel with “Blue Gal” (Life Technologies Inc., Gaithersburg) is used to allow easy identification and isolation of gal-expressing clones, which produce blue-stained plaques. A detailed description of a “plaque assay” of this type can also be found in the user's guide for insect cell culture and baculovirology distributed by Life Technologies Inc., Gaithersburg, Md., pages 9-10. After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later, the supernatants of these culture dishes are harvested and then they are stored at 4 C. The recombinant virus is called V-HSF.

To verify the expression of the HSF gene, Sf9 cells are grown in Grace's medium supplemented with 10% heat inactivated FBS. The cells are infected with the recombinant baculovirus V-HSF at a multiplicity of infection (“MOI”) of about 2. Six hours later the medium is removed and is replaced with SF900 II medium minus methionine and cysteine (available from Life Technologies Inc., Rockville, Md.). If radiolabeled proteins are desired, 42 hours later, 5 μCi of ³⁵S-methionine and 5 μCi ³⁵S-cysteine (available from Amersham) are added. The cells are further incubated for 16 hours and then they are harvested by centrifugation. The proteins in the supernatant as well as the intracellular proteins are analyzed by SDS-PAGE followed by autoradiography (if radiolabeled). Microsequencing of the amino acid sequence of the amino terminus of purified protein may be used to determine the amino terminal sequence of the mature protein and thus the cleavage point and length of the secretory signal peptide.

### EXAMPLE 3

### Cloning and Expression in Mammalian Cells

A typical mammalian expression vector contains the promoter element, which mediates the initiation of transcription of mRNA, the protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as PSVL and PMSG (Pharmacia, Uppsala, Sweden), pRSVcat (ATCC 37152), pSV2dhfr (ATCC 37146) and pBC12MI (ATCC 67109). Mammalian host cells that could be used include, human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded protein. The DHFR (dihydrofolate reductase) marker is useful to S develop cell lines that carry several hundred or even several thousand copies of the gene of interest. Another useful selection marker is the enzyme glutamine synthase (GS) (Murphy et al., Biochem J. 227:277-279 (1991); Bebbington et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

The expression vectors pC1 and pC4 contain the strong promoter (LTR) of the Rous Sarcoma Virus (Cullen et al, Molecular and Cellular Biology, 438-447 (March, 1985)) plus a fragment of the CMV-enhancer (Boshart et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

### Example 3(a)

### Cloning and Expression in COS Cells

The expression plasmid, pHSF HA, is made by cloning a cDNA encoding HSF into the expression vector pcDNA3 (Invitrogen, Chatsworth, Calif.). The expression vector pcDNA3amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin protein described by Wilson et al., Cell 37:767 (1984). The fusion of the HA tag to the target protein allows easy detection and recovery of the recombinant protein with an antibody that recognizes the HA epitope. pcDNA3 contains, in addition, the selectable neomycin marker.

A DNA fragment encoding HSF is cloned into the polylinker region of the vector so that recombinant protein expression is directed by the CMV promoter. The plasmid construction strategy is as follows. The HSF cDNA of the deposited clone is amplified using primers that contain convenient restriction sites, much as described above for construction of vectors for expression of HSF in E. coli. Suitable primers include the following, which are used in this example. The 5′ primer has the sequence: 5′-CGTCTAGACGCGGCCGCCACCCCACCATGCTC-3′ (SEQ ID NO:8), containing the underlined Xba I site, a Kozak sequence and 24 bases corresponding to nucleotides 48-71 in SEQ ID NO:1, including an AUG start codon. If no HA tag is used, the 3′ primer has the sequence 5′-TGGGTCTAGACCATGGCCACTAGAAGATG-3′ (SEQ ID NO:9), containing the underlined Xba I site and 19 bases reverse and complementary to nucleotides 1196-1214 of SEQ ID NO:1. If an HA tag is used, the 3′ primer has the sequence 5′-TGGGTCTAGACCATGGCCACTAAGCGTAGTCTGGGACGTCGTATGGGTAGAAGATG-3′ (SEQ ID NO:10), containing the underlined Xba I site and 12 bases reverse and complementary to nucleotides 1203-1214 in SEQ ID NO:1.

The PCR amplified DNA fragment and the vector, pcDNA3/Amp, are digested with Xba I and then ligated. The ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the HSF-encoding fragment.

For expression of recombinant HSF, COS cells are transfected with an S expression vector, as described above, using DEAE-DEXTRAN, as described, for instance, in Sambrook et al., Molecular Cloning: a Laboratory Manual, Cold Spring Laboratory Press, Cold Spring Harbor, N.Y. (1989). Cells are incubated under conditions for expression of HSF by the vector.

Expression of the HSF-HA fusion protein is detected by radiolabeling and immunoprecipitation, using methods described in, for example Harlow et al., Antibodies: A Laboratory Manual, 2nd Ed.; Cold Spring Harbor Laboratory Press, Cold Spring Harbor, N.Y. (1988). To this end, two days after transfection, the cells are labeled by incubation in media containing ³⁵S-cysteine for 8 hours. The cells and the media are collected, and the cells are washed and lysed with detergent-containing RIPA buffer: 150 mM NaCl, 1% NP-40, 0.1% SDS, 0.5% DOC, 50 mM TRIS, pH 7.5, as described by Wilson et al. cited above. Proteins are precipitated from the cell lysate and from the culture media using an HA-specific monoclonal antibody. The precipitated proteins then are analyzed by SDS-PAGE and autoradiography. An expression product of the expected size is seen in the cell lysate, which is not seen in negative controls.

### Example 3(b)

### Cloning and Expression in CHO Cells

The vector pC4 is used for the expression of HSF protein. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., Kellems, R. M., Bertino, J. R., and Schimke, R. T., 1978, J Biol. Chem. 253:1357-1370, Hamlin, J. L. and Ma, C., 1990, Biochem. et Biophys. Acta, 1097:107-143, Page, M. J. and Sydenham, M. A., 1991, Biotechnology 9:64-68). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified 0 gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al, Molecular and Cellular Biology, March 1985:438-447) plus a fragment isolated from the enhancer of the immediate early gene of human cytomegalovirus (CMV) (Boshart et al., Cell 41:521-530 (1985)). Downstream of the promoter are BamHi, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human P-actin promoter, the SV40 early or late promoters or the long terminal repeats from other retroviruses, e.g., HIV and HTLVI. Clontech's Tet-Off and Tet-On gene expression systems and similar systems can be used to express the HSF protein in a regulated way in mammalian cells (Gossen, M., & Bujard, H., 1992, Proc. Natl. Acad. Sci. USA 89: 5547-5551). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with the restriction enzyme Xba I and then dephosphorylated using calf intestinal phosphatase by procedures known in the art. The vector is then isolated from a 1% agarose gel.

The DNA sequence encoding the complete HSF protein including its leader sequence is amplified using PCR oligonucleotide primers corresponding to the 5′ and 3′ sequences of the gene. The 5′ primer has the sequence 5′5′-CGTCTAGACGCGGCCGCCACCCCACCATGCTC-3′ (SEQ ID NO:8), containing the underlined Xba I site, an efficient signal for initiation of translation in eukaryotes, as described by Kozak, M., J. Mol. Biol. 196:947-950 (1987), and 24 bases corresponding to nucleotides 48-71 in SEQ ID NO:1, including an AUG start codon. If no HA tag is used, the 3′ primer has the sequence 5′-TGGGTCTAGACCATGGCCACTAGAAGATG-3′ (SEQ ID NO:9), containing the underlined Xba I site and 19 bases reverse and complementary to nucleotides 1196-1214 of SEQ ID NO: 1.

The amplified fragment is digested with the endonuclease Xba I and then purified again on a 1% agarose gel. The isolated fragment and the dephosphorylated vector are then ligated with T4 DNA ligase. E. coli HB 101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. 5 μg of the expression plasmid pC4 is cotransfected with 0.5 μg of the plasmid pSV2-neo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM 100 nM 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reverse phase HPLC analysis.

### EXAMPLE 4

### Tissue Distribution of HSF Protein Expression

Results of Northern analyses have been negative. However, results from database analyses suggest that HSF is expressed in activated neutrophils.

It will be clear that the invention may be practiced otherwise than as particularly described in the foregoing description and examples.

Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. An isolated nucleic acid molecule comprising a polynucleotide having a nucleotide sequence at least 95% identical to a sequence selected from the group consisting of:
(a) a nucleotide sequence encoding the HSF polypeptide having the amino acid sequence at positions from about −26 to about 353 in SEQ ID NO:2;
(b) a nucleotide sequence encoding the HSF polypeptide having the amino acid sequence at positions from about −25 to about 353 in SEQ ID NO:2;
(c) a nucleotide sequence encoding the amino acid sequence at positions from about 1 to about 353 in SEQ ID NO:2;
(d) a nucleotide sequence encoding the HSF polypeptide having the complete amino acid sequence encoded by the cDNA clone contained in ATCC Deposit No. 97731;
(e) a nucleotide sequence encoding the mature HSF polypeptide having the amino acid sequence encoded by the cDNA clone contained in ATCC Deposit No. 97731; and
(f) a nucleotide sequence complementary to any of the nucleotide sequences in (a), (b), (c), (d) or (e).

2. An isolated nucleic acid molecule comprising a polynucleotide which hybridizes under stringent hybridization conditions to a polynucleotide having a nucleotide sequence identical to a nucleotide sequence in (a), (b), (c), (d) or (e) of claim 1, wherein said polynucleotide which hybridizes does not hybridize under stringent hybridization conditions to a polynucleotide having a nucleotide sequence consisting of only A residues or of only T residues.

3. An isolated nucleic acid molecule comprising a polynucleotide which encodes the amino acid sequence of an epitope-bearing portion of a HSF polypeptide having an amino acid sequence in (a), (b), (c), (d) or (e) of claim 1.

4. The isolated nucleic acid molecule of claim 3, which encodes an epitope-bearing portion of a HSF polypeptide selected from the group consisting of: a polypeptide comprising amino acid residues from about −26 to about −1 in SEQ ID NO:2; a polypeptide comprising amino acid residues from about 1 to about 26 in SEQ ID NO:2; a polypeptide comprising amino acid residues from about 56 to about 90 in SEQ ID NO:2; a polypeptide comprising amino acid residues from about 94 to about 106 in SEQ ID NO:2; a polypeptide comprising amino acid residues from about 112 to about 137 in SEQ ID NO:2; a polypeptide comprising amino acid residues from about 146 to about 181 in SEQ ID NO:2; a polypeptide comprising a mino acid residues from about 191 to about 222 in SEQ ID NO:2; a polypeptide comprising amino acid residues from about 257 to about 266 in SEQ ID NO:2; a polypeptide comprising amino acid residues from about 293 to about 304 in SEQ ID NO:2; and a polypeptide comprising amino acid residues from and about 311 to about 351 in SEQ ID NO:2.

5. An isolated nucleic acid molecule comprising a polynucleotide having a sequence at least 95% identical to a sequence selected from the group consisting of:
(a) the nucleotide sequence of a fragment of the sequence shown in SEQ ID NO:1, wherein said fragment comprises at least 50 contiguous nucleotides of SEQ ID NO:1, provided that said nucleotide sequence is not SEQ ID NO:11, SEQ ID NO:12, SEQ ID NO:13, SEQ ID NO:14, SEQ ID NO:15, or any subfragment thereof; and
(b) a nucleotide sequence complementary to a nucleotide sequence in (a).

6. A method for making a recombinant vector comprising inserting an isolated nucleic acid molecule of claim 1 into a vector.

7. A recombinant vector produced by the method of claim 6.

8. A method of making a recombinant host cell comprising introducing the recombinant vector of claim 7 into a host cell.

9. A recombinant host cell produced by the method of claim 8.

10. A recombinant method for producing a HSF polypeptide, comprising culturing the recombinant host cell of claim 9 under conditions such that said polypeptide is expressed and recovering said polypeptide.

11. An isolated HSF polypeptide having an amino acid sequence at least 95% identical to a sequence selected from the group consisting of:
(a) amino acid residues from about −26 to about 353 in SEQ ID NO:2;
(b) amino acid residues from about −25 to about 353 in SEQ ID NO:2;
(c) amino acid residues from about 1 to about 353 in SEQ ID NO:2;
(d) the amino acid sequence of the HSF polypeptide having the complete amino acid sequence encoded by the cDNA clone contained in ATCC Deposit No. 97731;
(e) the amino acid sequence of the mature HSF polypeptide having the amino acid sequence encoded by the cDNA clone contained in ATCC Deposit No. 97731; and
(f) the amino acid sequence of an epitope-bearing portion of any one of the polypeptides of (a), (b), (c), (d) or (e).

12. An isolated polypeptide comprising an epitope-bearing portion of the HSF protein, wherein said portion is selected from the group consisting of: a polypeptide comprising amino acid residues from about −26 to about −1 in SEQ ID NO:2; a polypeptide comprising amino acid residues from about 1 to about 26 in SEQ ID NO:2; a polypeptide comprising amino acid residues from about 56 to about 90 in SEQ ID NO:2; a polypeptide comprising amino acid residues from about 94 to about 106 in SEQ ID NO:2; a polypeptide comprising amino acid residues from about 112 to about 137 in SEQ ID NO:2; a polypeptide comprising amino acid residues from about 146 to about 181 in SEQ ID NO:2; a polypeptide comprising amino acid residues from about 191 to about 222 in SEQ ID NO:2; a polypeptide comprising amino acid residues from about 257 to about 266 in SEQ ID NO:2; a polypeptide comprising amino acid residues from about 293 to about 304 in SEQ ID NO:2; and a polypeptide comprising amino acid residues from and about 311 to about 351 in SEQ ID NO:2.

13. The isolated polypeptide of claim 11, which is produced or contained in a recombinant host cell.

14. The isolated polypeptide of claim 13, wherein said recombinant host cell is mammalian.

15. An isolated nucleic acid molecule comprising a polynucleotide encoding an HSF polypeptide wherein, except for one to fifty conservative amino acid substitutions, said polypeptide has a sequence selected from the group consisting of:
(a) a nucleotide sequence encoding the HSF polypeptide having the amino acid sequence at positions from about −26 to about 353 in SEQ ID NO:2;
(b) a nucleotide sequence encoding the HSF polypeptide having the amino acid sequence at positions from about −25 to about 353 in SEQ ID NO:2;
(c) a nucleotide sequence encoding the amino acid sequence at positions from about 1 to about 353 in SEQ ID NO:2;
(d) a nucleotide sequence encoding the HSF polypeptide having the complete amino acid sequence encoded by the cDNA clone contained in ATCC Deposit No. 97731;
(e) a nucleotide sequence encoding the mature HSF polypeptide having the amino acid sequence encoded by the cDNA clone contained in ATCC Deposit No. 97731; and
(f) a nucleotide sequence complementary to any of the nucleotide sequences in (a), (b), (c), (d) or (e).

16. An isolated HSF polypeptide wherein, except for one to fifty conservative amino acid substitutions, said polypeptide has a sequence selected from the group consisting of:
(a) amino acid residues from about −26 to about 353 in SEQ ID NO:2;
(b) amino acid residues from about −25 to about 353 in SEQ ID NO:2;
(c) amino acid residues from about 1 to about 353 in SEQ ID NO:2;
(d) the amino acid sequence of the HSF polypeptide having the complete amino acid sequence encoded by the cDNA clone contained in ATCC Deposit No. 97731;
(e) the amino acid sequence of the mature HSF polypeptide having the amino acid sequence encoded by the cDNA clone contained in ATCC Deposit No. 97731; and
(f) the amino acid sequence of an epitope-bearing portion of any one of the polypeptides of (a), (b), (c), (d) or (e).

17. An isolated antibody that binds specifically to a HSF polypeptide of claim 11.

18. An isolated antibody that binds specifically to a HSF polypeptide of claim 12.


SEQUENCE LISTING (15 sequences)

SEQ ID NO: 1 - DNA (cDNA), 1545 bp; double-stranded, linear
  CDS 66..1202
  mat_peptide 144..1202
  sig_peptide 66..141
cgggcttcgggtcggtgcaaggcaggcgcacggggaagggcgcgccgcgcggccgccaccccaccatgctcaagcgctgcggccggcgcctgctgctggcgctggcgggcgcgctgctcgcctgcctgctggtgctcaccgccgacccgccgccgcctccactgcccgccgagcgcggccggcgcgcgctgcgcagcctggcgggccccgcgggggctgccccggcgcccgggctgggggcggcggcggcggcgcccggggcgctggtccgcgacgtgcacagtctgtccgagtacttcagcctgctcacccgcgcgcgcagagatgcgggcccgccgcccggggctgccccccgccccgccgacggccacccgcgccccctggccgagccgctcgcgccccgagacgtcttcatcgctgtcaagaccaccaaaaagttccaccgcgcgcgcctcgacctgctgctggagacctggatctcgcgccacaaggagatgacgttcatcttcactgacggggaagatgaggccctggccaggcacacgggcaacgtggtcatcacaaactgctcggccgcccacagccgccaggcgctgtcctgcaagatggccgtggagtatgaccgcttcatcgagtccggcaggaagtggttctgccacgtggacgatgacaactacgtcaacctgcggaccctgctgcgactgctggccagctacccgcacacgcgggacgtctacgtcggcaagcccagcctggacaggcccatccaggccatggagcgggtcagcgagaacaaggtgcgtcctgtccacttctggtttgccacgggcggcgctggcttctgcatcagccgtgggctggctctgaagatgagcccgtgggccagcgggggtcacttcatgaatacggctgagcggatccggctgcctgatgactgcaccatcggctacatcgtggaggccctgctgggtgtgcccctcatccgcagcggcctcttccactcccacctggagaacctgcagcaggtgcccacctcggagctccacgagcaggtgacgctgagctacggtatgtttgaaaacaagcggaacgccgtccacgtgaaggggcccttctcggtggaggccgacccatccaggttccgctccatccactgccacctgtacccggacacaccctggtgtccccgcactgccatcttctagtggccatggctgagacccaatccctgggcgcccctggtatccaaagggcccagggaccctgttgcgctgccctggcctcggcattcgaggctcccctagggccgtgcctgtgcgtgtgcgtgtgcgtgtgtgtgtgtgtgtactgcatgcccacccgggtagcaggctgctgggcagttctgctctgtggaggggcgggcaccagcgccacttatgtgcctctgctccgagggccagtggtatggagggtctgcttggaggaaggatttgtgtgtcggaggacactccgagggcaattctgttaggatttgtggatctttctacagctacggggctccgg

SEQ ID NO: 2 - protein, 379 aa
MLKRCGRRLLLALAGALLACLLVLTADPPPPPLPAERGRRALRSLAGPAGAAPAPGLGAAAAAPGALVRDVHSLSEYFSLLTRARRDAGPPPGAAPRPADGHPRPLAEPLAPRDVFIAVKTTKKFHRARLDLLLETWISRHKEMTFIFTDGEDEALARHTGNVVITNCSAAHSRQALSCKMAVEYDRFIESGRKWFCHVDDDNYVNLRTLLRLLASYPHTRDVYVGKPSLDRPIQAMERVSENKVRPVHFWFATGGAGFCISRGLALKMSPWASGGHFMNTAERIRLPDDCTIGYIVEALLGVPLIRSGLFHSHLENLQQVPTSELHEQVTLSYGMFENKRNAVHVKGPFSVEADPSRFRSIHCHLYPDTPWCPRTAIF

SEQ ID NO: 3 - protein, 375 aa
MLKNWGKKLLLSIVGATLTCLLVLVVDQQSRHMLETQSDHEPGSAAAVHLRADLDPANPGDGGDPANSAQDSGTFSAYFNKLTRVRRDVEQVAAPSKDSAAPEEDITANDVFIAVKTTKKFHRSRMDLLMDTWISRNKEQTFIFTDGEDEELQKKTGNVESTNCSAAHSRQALSCKMAVEYDKFIESDKKWFCHVDDDNYVNVRTLVKLLSRYSHTNDIYIGKPSLDRPIQATERISESNMRPVNFWFATGGAGFCISRGLALKMSPWASGGHFMNTAEKIRLPDDCTIGYIIESVLGVKLIRSNLFHSHLENLHQVPQSEIHNQVTLSYGMFENKRNAILMKGAFSVEEDPSRFRSVHCLLYPDTPWCPWKAAY

SEQ ID NO: 4 - DNA (cDNA), 29 bp; single-stranded, linear
caccgtcgacccgccgccgcctccactgc

SEQ ID NO: 5 - DNA (cDNA), 37 bp; single-stranded, linear
ggtctaagctttggccattagaagatggcagtgcggg

SEQ ID NO: 6 - DNA (cDNA), 34 bp; single-stranded, linear
gcgtctagaccgccatcatgctcaagcgctgcgg

SEQ ID NO: 7 - DNA (cDNA), 22 bp; single-stranded, linear
gtaatacgactcactatagggc

SEQ ID NO: 8 - DNA (cDNA), 32 bp; single-stranded, linear
cgtctagacgcggccgccaccccaccatgctc

SEQ ID NO: 9 - DNA (cDNA), 29 bp; single-stranded, linear
tgggtctagaccatggccactagaagatg

SEQ ID NO: 10 - DNA (cDNA), 56 bp; single-stranded, linear
tgggtctagaccatggccactaagcgtagtctgggacgtcgtatgggtagaagatg

SEQ ID NO: 11 - DNA (cDNA), 418 bp; both-stranded, linear
tggtttgcnacgggnggcgctggcttctncatcanccgtgggctggctctaaagatgagcccgtgggccagcgggggtnacttcatgaatacggctgagcggatccggcngcctgatgactgcaccatcggctacatcgtggaggccctgctgggtgtgcccctnatccgcagcggcctnttccactcccacctggagaacctgcagcaggtgcccacctcggagctccacggagcaggtnacgctgagcttacggtattttttgaaaaaaagcgggaacgccgttccacgtnaaagggggccttttttnggttgggagggccgacccattccaggtttccgtttncattccanttgccaacttttaaccngggaaaaaaccttggtntttccccgaaattgnaattttttaggttgg

SEQ ID NO: 12 - DNA (cDNA), 443 bp; both-stranded, linear
ggcacgagtgaagggctgcctgctgaaggccgattttctccttccagacgttcatcttcactgacggggaagatgaggccctggccaggcacacgggtgagccctggacttggggcggganggggcccaagcctccatccagagccgaacgctccccctccagtctccctgcccctctgggccgagaagtcaccaagggcaggacanggagggcagtttactcatggggtttgctccatgccccggcccaacactcgggncccccaattctcatgcaaatgaagcccattcagccccccgggttcctttgagccaagcagcggcaaaagtggcggttctggnaaaagtgctgattggcggggcggggcgccttgaagggttggttcggaagtcaaagccaagcccgagtaagttgggagtangcgccggccagttaanaaggg

SEQ ID NO: 13 - DNA (cDNA), 513 bp; both-stranded, linear
ctcgagtccgggtgcaggtggcaatggacagagcggaacctggatgggtcagcttccacagagaatggtcccttgatgtgcactgcgttccgcttgttctcaaacatgccatagctcagggtcacctgctcatgaagctcggtggtgggcacctgctgcaggttctctaggtgggagtggaagaggccgctccggatgaggggtacacccagcagagcctctacaatgtagccaatggtgcagtcatcggggagccggatgcgctctgccgtgctcatgaagtgtcctccactggcccatgggccatctttagggccagccctcggctgatgcagaagccagctcctccggtggcaaaccaaaagtggacaggtctcactttgtgctcgctgatccgttctgtggcctggatgggcctgtccaggctgggcttgcgatgtacacgtcttgggtgtggggatagctggccaggaagcgcagcagcgcccggaagttgacgtagttgtcatcatc

SEQ ID NO: 14 - DNA (cDNA), 352 bp; both-stranded, linear
gcgccccctggccgagccgctncgcgccccgagacgtcttcatcgctgtcaagaccaccaaaaagttccaccgcgcgcgcctcgacctgctgctggagacctggatctcgcgccacaaggagatgacgttcatcttcactgacggggaagatgaggccctggcaggcacacgggcaacgtggtcatcacaaactgctcggccgcccacagccgccagcttgnttcctgcaagatggccgtggagtatgaccgcttcatcgagtccggcagaagtggttctgccacgtggacgatgacaactacgtcaacctgcgggccctgctncggctgctggccagctgacccgcacacg

SEQ ID NO: 15 - DNA (cDNA), 449 bp; both-stranded, linear
acaggtctcactttgtgctcgctgatccgttctgtggcctggatgggctgtccagctgggcttgcgatgtcacgtcttgggtgtggggatagctggccaggagccgcagcagcgcccggagttgacgtagttgtcatcatccacgtggcagaaccacttcttcccagactcaatgaatcggtcatactccacagccatcttgcaggacagagcctggcgctgtgggccgaggagcagttggtgagcaccacattgcctgtgagcttggccagagcttcgtcctccccatcagtgaagatgaacgtcatctccttgtggcgcgagatccaggtctcgaacagcagatcgagccgcgcgcggtgaaactttctggtggtcttgacggcgatgaagacgtcgcgaggggacagaactttcggcgggggacgcggatgggcgtcgccctgcga